(19) United States
(12) Patent Application Publication
(10) Pub. No.: US 2008/0260668 A1
(43) Pub. Date: Oct. 23, 2008

(54) Method for Preparing a Compound for Drawing a Non-Permanent Tattoo and a Method of Using Said Compound

(76) Inventor: Daniela Vidalenc, Sao Paulo (BR)

Correspondence Address: Ronald R Santucci;Frommer Lawrence Haug LLP, 745 Fifth Avenue, New York, NY, 10151, US

(21) Appl. No.: 11/579,250

(22) PCT Filed: May 4, 2005
(86) PCT No.: PCT/BR2005/000070

§ 371 Date: May 11, 2007

(30) Foreign Application Priority Data
May 4, 2004 (BR) PI 0402011-1

(51) Int. Cl. A61K 8/97; A61Q 99/00

(52) U.S. Cl. 424/63

(57) ABSTRACT

The present invention refers to a method for preparing of a compound for drawing a non-permanent tattoo from the “jenipapo” fruit (Genipa americana). A certain amount of juice from the “jenipapo” fruit (Genipa americana) is provided, into which a thickener is added for forming a substantially creamy compound, which receives extracts from the seeds of grapefruit, rosemary, olives leaves, E vitamin, citric acid, ascorbic acid and vegetable dyes as additives, as well as colorincreasing compounds for increasing the intensity of its color. The compound is stored in sealed packages for future use to make free hand designs and to make adhesive substrates with voided designs and protective backings.

### BACKGROUND OF THE INVENTION

1. Field of the Invention

The present invention generally refers to the cosmetic industry and, more specifically, to methods for extracting, preserving, industrializing and applying a natural dye in raw state, said natural dye originating from the fruit of the Genipa Americana and being used for topic application upon the skin in such a manner that when in contact with the skin the dye reacts with the biochemical components of the skin, complexing therewith it and fixing itself onto the skin.

2. Description of Prior Art

As it is known by those skilled in the art, the fruit of the Genipa Americana (“jenipapo”) has been used since before the discovery of Brazil circa 1500 by a number of native Brazilian indigenous communities, including those living in areas near or at the borders of the country, for painting pictures and/or for designing tattoos.

The traditional method of painting pictures or designing tattoos using the fruit of the Genipa Americana (“jenipapo”) comprised extracting the juice from the fruit by picking it up while still not ripe and masticating its inner part with the teeth, the chewed mass being spewed into pots which were then heated to boil the resulting mixture.

The solid residues and impurities were separated from the liquid by an extremely rudimentary filtering method, thereby forming a dye which could be applied upon the skin and which reacted with the skin to define lines and traces on top of the region where it was applied.

The design was formed as a result of the chemical reaction between the unripe juice of the Genipa Americana, commonly known as “genipina”, with certain skin substances, with the result appearing rather slowly, said design also depending on certain correlated conditions such as the quality of the fruit, the temperature of the body and other environmental conditions.

According to the traditions of some indigenous communities, the liquid can be additionally mixed or blended with coal, in order to form a paste which allows the design being formed upon the skin to be visualized during the very moment of its application.

This dye, and/or the paste formed by mixing it with coal, is kept and reused a number of times until the dye eventually degrades and no longer function for the purposes of coloring the skin, the main causes of the coloring loss of strength being the oxidation, the micro biotic degradation and the sensibility of the dye to variations in temperature.

Due to this deterioration by oxidation by micro biotic degradation or due to the variations in the temperature of the liquid dye or of the paste formed by mixing it with coal simply makes it not viable to be used as a material for the colouring bases in the cosmetics industry.

Another problem found when using this dye, or the paste formed by it, for the making of designs or tattoos is the very high trickling or smearing capacity of the said dye or paste. Due to this trickling or smearing capacity, a trace or line having an original width of 1 mm may expand up to at least 4 mm, depending of the part of the body upon which the dye is applied.

An additional problem which makes the use of the said dye not viable is the final result of the liquid reaction with the skin, because of the fact that the intensity of its bluish black colour greatly depends of the body part where upon the dye is applied. The knees, feet, hands, elbows and the buttocks regions react more intensely with the dye extract from the unripe fruit thus producing a darker coloration than the regions like the arms, abdomen and legs.

Considering all the above mentioned, it is clear that the dyes based in the “jenipapo” fruit (Genipa americana) known in the state of the art show various disadvantages, and amongst them stands out the large thickness of the designs traces or lines, caused by the trickling or smearing capacity, the said large thickness making impossible to draw and to design fine lines and/or traces.

Also, due to the trickling and/or the smearing capacity, small lines derive from the main traces, or else they spread around themselves, in accordance to the dye penetration in the skin, thus producing the phenomena known as “fishbone effect”. Even if it is possible to avoid the trickling and/or the smearing factor, an enormous artistic ability would be necessary to be able to make good quality designs and drawings.

The application of the dye is typically carried out with the fingers, which makes them dirty and stained, or with a fine point stick, which makes it impossible to produce curving designs and, at the same time, rather detailed designs.

It is not possible to control the colour intensity when applying the dye upon the skin, and it is practically not viable to create a protection against accidents which may stain the design or even other parts of the body.

There is a problem of a very low production rate and yielding during the extraction of the juice and there is a great deal of effort to produce it, not to mention the impossibility of storing the unripe fruit, which altogether generates a full dependency on the natural cycles.

There is no control over the “jenipapo” fruit (Genipa americana) time of pigmentation showing after its application upon the skin.

### SUMMARY OF THE INVENTION

The purpose of the present invention is to provide a method for the preparing of a compound for a non permanent tattoo and its respective form of using, solving all the inconvenience found in the present state of the art. The said objectives are reached by an innovative manner to produce a natural compound based on the “jenipapo” fruit (Genipa americana), which is capable of keeping itself stable by a prolonged duration of time, thus making possible the production of designs without the trickling or smearing of it onto the skin, through a relatively easy application method, which is free of errors.

According to the present invention, this purpose is reached by the provision of a method for the preparing of a compound for drawing non-permanent tattoo, which comprises the steps of:

providing a certain amount of a juice extracted from the fruit of the Genipa Americana plant (“genipapo”);

adding a thickener to said juice, said thickener being added in an amount sufficient to form a substantially creamy mixture;

adding extracts from the seeds of grapefruit (Citrus decumana), rosemary, olive leaves, E vitamin, citric acid, ascorbic acid, and vegetable dyes to the said creamy mixture, said extracts acting as both preserving and antioxidant agents;

adding color-increasing components to said creamy mixture to increase the intensity of the compound; e

saving the creamy compound in a sealed package for posterior use.

Still according to the present invention, the step of providing a certain amount of a juice extracted from the fruit of the Genipa Americana plant (“genipapo”) comprises the steps of:

picking up the fruits from the Genipa Americana plant while still unripe;

washing the fruits with a jet of water at a sufficient pressure in order to totally clean the fruit without damaging it;

sterilizing the clean fruits with a solution of chlorine and alcohol;

freezing the washed and sterilized fruits for a period of time;

unfreezing the fruits;

pressing the unfrozen fruits in order to obtain a juice from the said fruits;

filtering the juice obtained from pressing said unfrozen fruits in order to eliminate any solid particles in it;

passing a bubbling flow of liquid nitrogen through said filtered juice to remove the oxygen from the juice while simultaneously cooling it with the liquid nitrogen;

allowing the filtered, nitrogen-filled, cooled juice to rest until it returns to room temperature, whilst concurrently liberating the nitrogen and stopping the increase of oxygen by competition; and

storing the juice in a package non-permeable to oxygen.

Preferably, the vegetable dyes used are selected from the group consisting of Lawsonia inermis, Curcuma longa and/or mixtures thereof, said dyes allowing for the lines of the tattoo to be visualized at the very moment the compound is applied upon the skin.

The rosemary extract is added in the form of an aqueous and oily extracts and said color-increasing components are selected from the group consisting of soy amino acids; hydrolyzed soy; arginina; phenylanine; metionine; lysine; bromeline; BHT; and BHA.

Additionally, the creamy compound is stored in toothpaste-like aluminum containers having a discharge opening at one end and a feeding opening at the opposite end, the discharge opening and the feeding opening being sealed to prevent any intake of oxygen.

Preferably said toothpaste-like aluminum containers are provided with a nozzle of the ophthalmic point type, having a small opening at its distal end, to allow the drawing of free hand made designs directly over the skin.

The present invention also refers to a method of using the compound for drawing a non-permanent tattoo, said method comprising the steps of:

providing a substrate in the shape of a mask having a plurality of void areas defining a pattern or design to be formed upon the skin, the rest of the substrate covering and protecting the areas which are not to be touched by the compound; and

applying said compound for drawing a non-permanent tattoo over said substrate to contact only the areas of the skin not covered by said substrate mask to color the skin.

Said substrate mask being a flexible plastic plate, impervious and waterproof, having a non-toxic glue applied to one of its surfaces to adhere to the skin and being made from a impervious material to the coloring compound to prevent it from passing through the material forming the substrate, the non-toxic glue having a sufficiently strong adhesive strength to the skin to stop the compound from trickling or smearing over the limits of the substrate or going under the substrate.

### BRIEF DESCRIPTION OF THE DRAWINGS

FIG. 1 shows a perspective view of an example of using the compound of the invention, wherein the said compound, conditioned in a tube like container with a fine point, is being applied upon the skin of a user through a voided area substrate.

### DETAILED DESCRIPTION OF THE PREFERRED EMBODIMENT

A currently preferred specific embodiment of the present invention is illustrated as an example in the annexed drawings and it will be next described in details. Nevertheless, it must be understood that although the present invention is susceptible to various modifications and alterations of forms and dimensions, the present description report has no intention at all of limiting the said invention to the particular forms and/or dimensions hereby described but, on the contrary, it encompasses all the said modifications and alternatives embodiments which are within the spirit and the scope of the invention, as defined by the annexed claims.

In accordance to the present invention, a method is provided for the preparing of a compound for the making of a non permanent tattoo based on the “jenipapo” fruit (Genipa americana).

Furthermore, according to the present invention the extraction and the storing of the juice makes it possible for a greater production and yielding of the said juice, thus minimizing its deterioration.

The unripe “jenipapo” fruit is frozen and, at the time of its use, the said fruit is washed with a jet of water strong enough to remove any lichen and any other impurities from the fruit's peel. The fruit is additionally sterilized with a solution of chlorine and alcohol, the said solution being used to kill fungi, bacteria and, further more, it has a great potential for killing the micro bacteria which are abundant in the micro flora from forests in general.

After being washed and sterilised, the still whole fruits are unfrozen and pressed in a hydraulic press specially adapted (steel mesh with holes for pouring into a collecting tray isolated with plastic material), to allow for the pouring of the juice without any loss of the said fruit and of the said juice.

The freezing of the fruits allow for the formation of ice crystals which perforate the cellular material of the whole fruit. This very perforation makes the out flow for the liquid juice much easier and the fruit remains, in a general manner, less bloated and rigid thus facilitating the pressing of it. As a consequence the yielding is much superior then the one obtained and achieved by the traditional indigenous form.

On the other hand, the washing and the sterilizing of the fruits allow for the use of the whole fruit without the need to reject the outer part whilst minimizing the possibility of infection and contamination by micro biotic agents of the peel.

The washing method therefore allows for the increase in production and contributes towards the method of minimizing the deterioration factor.

The pressing system is also much more efficient with regards to production and yielding than the chewing or the use of a pestle and mortar, apart from the fact it practically does not produce any solid residues so that a simple filtering procedure with nylon sieves may remove any solid waste which may have come apart.

The pressing with a plastic sealing allows for the coupling of gaseous nitrogen to maintain the extraction under inert atmosphere which stops the extract's oxidation and maintains its durability much more extensively, for the oxygen is the pigment's main deteriorating agent.

Once it is extracted, the liquid juice is bubbled with liquid nitrogen for 5 minutes, which reduces the extract temperature and, therefore, increases the amount of dissolved gas.

Still, with the natural heating of the juice whilst waiting for the making of the tattoo cream, the liquid supports less dissolved gas in a decreasing manner and therefore frees the nitrogen thus stopping the oxygen increase by competition.

It should be noted that the use of nitrogen gas in the extraction and in the storing of the juice is indeed a novelty regarding the blocking of the juice's oxidation.

For the making of the tattoo compound in the form of a cream according to the present invention, the nitrogen is bubbled in the interior of and above the mixture, so that the atmosphere be inert, that is, without oxygen, all of this method being carried out in perfectly aseptic conditions of both the equipment and the specialized personnel.

If compared to the indigenous form of preparing, these methods add value with regards to the durability of the tattoo cream, which makes it possible, together with another batch of measures, the industrializing and the commercializing of the said tattoo cream.

For the act of tattooing, the indigenous peoples use 100% of the juice of the unripe “jenipapo” fruit (Genipa americana) or, optionally, with the addition of a minimum amount of vegetable coal. The present invention technique allows for the limited use of the juice within a range of 10% up to 98% of coloring strength, to provide variable coloration intensities.

The method according to the present invention includes the use of “amigel”, a thickener which is capable of retaining the liquid in such a sufficient manner that a trace or line 1 mm wide remains with this exact measure, without any design misalignment or failure, even if there are distortions presented by the microscopic skin cracks.

This allows for the same compound to be used in temporary make-up for the eyelids and for the eyebrows, using a liquid aqueous extract in a liquid cream formulation, applied for instance with a delineating brush, of the eye delineator type.

In the indigenous people's form of application the trace or line is both wider as well as irregular by the fact that the liquid invades the veins of the skin's superficial cracks, in such a manner that the design lines periphery acquires the appearance of a “fishbone (the fishbone effect)”. With the use of thickeners of the polymer type (derived from starch, pectin, cellulose, powders from plants—because of the high content of pectin or cellulose or starch or the likes—gums, and others), one may reach this very effect of retention of the traced design.

The active substance which colors the skin, in the inventive compound, is the “genipina” and the correlated molecules such as the “geniposídio” (geniposideo) and the “genipínico” (genipinic) acid.

In accordance to the present invention, the compound, its preparing and its use involves: the extraction and the storing of the juice from the “jenipapo” fruit (Genipa americana), which is a common fruit in the Americas, found in tropical and sub-tropical areas, the preparing of a compound in the form of a tattoo cream, physically and chemically stable and of easy application.

Furthermore, the use of an appropriate package containing the compound, for the free design considering the fact one will be handling the very package for the actual designing procedure, stops the oxidation and the reflux of air after the said package has been opened, something which would otherwise degrade the compound.

The preparing of the invention's compound includes the addition of an extract from grapefruit seeds, which has bactericidal action and helps the preserving method, the addition of rosemary in the form of an aqueous and oily extract as a natural anti oxidizing agent, includes the addition of an extract from olive leaves as a bactericidal and anti oxidizing agent. Includes the addition of E vitamin as the formulation anti oxidizing agent as well as the skin's; includes the addition of citric and ascorbic acid which minimizes the compound oxidation. Some vegetable dyes are additionally added such as Lawsonia inermis, as well as Curcuma longa, so that the trace or line be visualized at the very moment of the application, thus avoiding the use of coal, as it occurs in the indigenous people's technique.

The compound allows for the use of co adjutants for the color intensity increase, such as soy amino acids, or hydrolyzed soy; arginina; phenylanine; metionine; lysine; bromeline; BHT; and BHA.

In the present invention, there is also a novelty with regards to the packaging of the compound: the use of aluminum tube like containers with nitrogen injection during the packaging guarantees an inert atmosphere and the non degradation of the compound. The aluminum tube like container allows for the compound to be used with the minimum inflow of oxygen after it has been opened due to the fact that there is no air reflux as there normally is with plastic tube like containers and the similar. In its forward end (the cream's exit part) the tube like container is sealed which stops the inflow of oxygen. The packaging is carried out in its posterior end part. The tube like container has a sealing polymer (DAREX) in its posterior end part which seals the tube like container thus stopping the inflow of oxygen. The tube like container has a small dimension ophthalmic point, which reduces the contact area with the oxygen, remaining sealed so that the oxygen does not inflow into the tube like container until the said tube like container is opened by the user.

Another novelty consists in the form of application of the tattooing compound, or tattooing cream, for the making of designs. The aluminum tube like container fine point makes it possible to produce the designs by free hand with the cream being pushed out in a gradual form as the pressure is applied to the tube like container's body part.

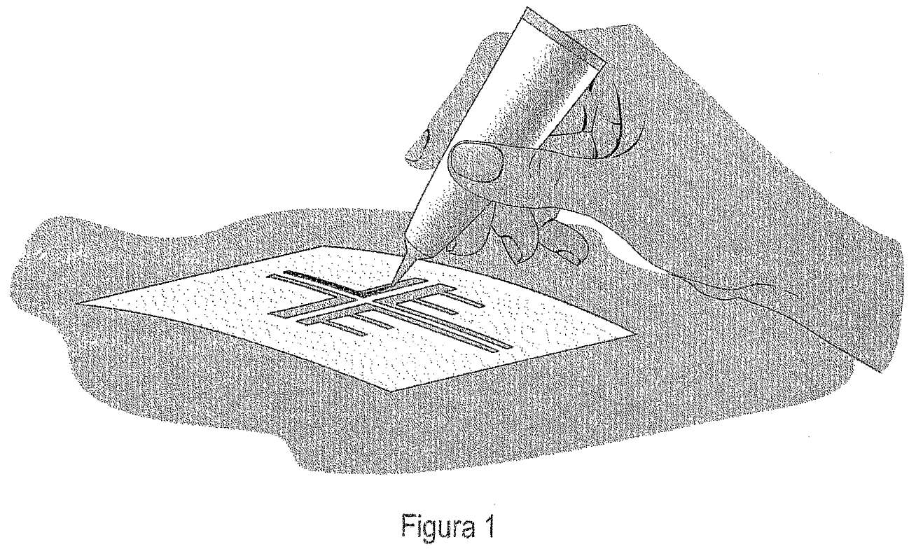

The compost form of using includes adhesive substrates, of the flexible plastic plate type, with the voided design to be tattooed; when the user detaches the substrate and places it on the skin, only the area to be tattooed (voided design) remains uncovered, with the rest of the substrate covering and protecting the other skin areas which should not be touched by the tattooing compound, as it is shown in FIG. 1.

The adhesive substrate is made of an impervious or waterproof material with a non toxic glue with a sufficiently strong adhesive strength so that the cream does not go beyond the limits of the substrate (be it through the adhesive material, be it under the adhesive due to the lack of glue or due to the adhesive's excessive elasticity). It is worth mentioning the fact that this method of using is new due to the unlimited variations of possible designs, easily made by the user, whom will only need to apply the cream with an application brush over the skin exposed parts. The tattooing compound forms a film of skin which is removed together with the adhesive substrate.

The compost form of using also includes, adhesive protecting backings with the following innovative functions: a protective backing to protect the tattooed area from eventual accidents which may dislocate the tattoo and cause it to be blurred, or from the applied cream being in contact with other body parts, staining them. In addition, the protective backing allows the cream to remain humid, therefore acting during the method of application and also allowing the area to remain in a higher temperature which increases the reaction capacity of the compound with the skin thus making possible an uniformity of the color intensity, independently of the area applied, with the result being visible within approximately 30 minutes after the removal of the adhesive substrate.

The innovative advantages of the invention in relation to the extraction and the storing are:
The use of the whole fruit, with the control of the juice's degradation;
The increase in production and yielding;
The absence of solid particles or residues;
The possibility of production throughout the year, with no seasonal dependency.

Finally, when compared to the presently known state of the art, the method for the preparing of a compound for making a non permanent tattoo according to the present invention provides a number of advantages, and amongst them stands out the possibility of its use in a concentration from 10% up to 95% of the juice in the tattoo cream, something which allows the obtaining of shades of a sort of a bluish colour which varies from a light blue shade up to shades of black and navy blue.

Further still, the compound according to the present invention offers a controlled expansion over the skin, thus eliminating the trickling and/or smearing factor.

Another batch of advantages are the possible industrialization of the tattoo cream, which physical-chemical stabilization can be reached by a prolonged duration of time, without mentioning the fact the designs can be visualized at the very moment they are being done, without the use of coal.

With regards to the method for the use of the compound for tattooing in accordance to the present invention, the compound packaging allows for the stanching of oxygen, reducing the contact with air, and facilitates the making of a free hand design.

Further still, the packaging and the adhesive backings do not dirt one's hands, do not stain the design, do not stain the undesirable areas, and provide the same strong color intensity in any part of the body.

Other advantages regarding the method for the preparing of a compound for the making of a non permanent tattoo according to the present invention are the possibility of carrying out designs with curves and fine details, with the sheer authenticity between the proposed design and the one set onto the skin, with a rather ease of application.

Having been described and illustrated the best presently contemplated form of carrying out the present invention, a number of modifications in its own form of carrying out will be promptly apparent to those skilled in the art. Therefore, it must be understood the fact that the present invention is not limited to the practical aspects of the embodiment presently preferred illustrated and described, and also that all such modifications and variations should be considered as being encompassed within the spirit and the scope of the invention, such as defined in the attached claims.

1. A method for preparing a compound for designing a non-permanent tattoo, said method comprising the steps of:
providing a certain amount of a juice extracted from the fruit of the Genipa Americana plant (“genipapo”);
adding a thickener to said juice, said thickener being added in an amount sufficient to form a substantially creamy mixture;
adding extracts from the seeds of grapefruit (Citrus decumana), rosemary, olive leaves, E vitamin, citric acid, ascorbic acid, and vegetable dyes to the said creamy mixture, said extracts acting as both preserving and antioxidant agents;
adding color-increasing components to said creamy mixture to increase the intensity of the compound; e
saving the creamy compound in a sealed package for posterior use.

2. The method for preparing a compound for designing a non-permanent tattoo according to claim 1, wherein the step of providing a certain amount of a juice extracted from the fruit of the Genipa Americana plant (“genipapo”) comprises the steps of:
picking up the fruits from the Genipa Americana plant while still unripe;
washing the fruits with a jet of water at a sufficient pressure in order to totally clean the fruit without damaging it;
sterilizing the clean fruits with a solution of chlorine and alcohol;
freezing the washed and sterilized fruits for a period of time;
unfreezing the fruits;
pressing the unfrozen fruits in order to obtain a juice from the said fruits;
filtering the juice obtained from pressing said unfrozen fruits in order to eliminate any solid particles in it;
passing a bubbling flow of liquid nitrogen through said filtered juice to remove the oxygen from the juice while simultaneously cooling it with the liquid nitrogen;
allowing the filtered, nitrogen-filled, cooled juice to rest until it returns to room temperature, whilst concurrently liberating the nitrogen and stopping the increase of oxygen by competition; and
storing the juice in a package non-permeable to oxygen.

3. The method for preparing a compound for designing a non-permanent tattoo according to claim 1, wherein the vegetable dyes used are selected from the group consisting of Lawsonia inermis, Curcuma longa and/or mixtures thereof.

4. The method for preparing a compound for designing a non-permanent tattoo according to claim 3, wherein said dyes allow for the lines of the tattoo to be visualized at the very moment the compound is applied upon the skin.

5. The method for preparing a compound for designing a non-permanent tattoo according to claim 1, wherein the rosemary extract is added in the form of an aqueous and oily extracts.

6. The method for preparing a compound for designing a non-permanent tattoo according to claim 1, wherein said color-increasing components are selected from the group consisting of soy amino acids; hydrolyzed soy; arginina; phenylanine; metionine; lysine; bromeline; BHT; and BHA.

7. The method for preparing a compound for designing a non-permanent tattoo according to claim 1, wherein the creamy compound is stored in toothpaste-like aluminum containers having a discharge opening at one end and a feeding opening at the opposite end.

8. The method for preparing a compound for designing a non-permanent tattoo according to claim 7, wherein the discharge opening in said container is sealed to prevent any intake of oxygen.

9. The method for preparing a compound for designing a non-permanent tattoo according to claim 7, wherein the feeding opening is mechanically sealed with a sealing polymer after the compound is fed into the container to prevent any intake of oxygen.

10. The method for preparing a compound for designing a non-permanent tattoo according to claim 7, wherein said toothpaste-like aluminum containers are preferably provided with a nozzle of the ophthalmic point type, having a small opening at its distal end.

11. The method for preparing a compound for designing a non-permanent tattoo according to claim 10, wherein the nozzle of the ophthalmic point type allows the drawing of free hand made designs directly over the skin.

12. A method of using the compound for drawing a non-permanent tattoo as described in claim 1, said method comprising the steps of:
providing a substrate in the shape of a mask having a plurality of void areas defining a pattern or design to be formed upon the skin, the rest of the substrate covering and protecting the areas which are not to be touched by the compound; and
applying said compound for drawing a non-permanent tattoo over said substrate to contact only the areas of the skin not covered by said substrate mask to color the skin.

13. The method of using of the compound for drawing a non-permanent tattoo according to claim 12, wherein said substrate mask is adhesive to the skin.

14. The method of using of the compound for drawing a non-permanent tattoo according to claim 13, wherein said adhesive substrate is a flexible plastic plate, impervious and waterproof, having a non-toxic glue applied to one of its surfaces.

15. The method of using of the compound for drawing a non-permanent tattoo according to claim 14, wherein said substrate mask is made from a impervious material to the coloring compound to prevent it from passing through the material forming the substrate.

16. The method of using of the compound for drawing a non-permanent tattoo according to claim 14, wherein the non-toxic glue has a sufficiently strong adhesive strength to the skin to stop the compound from trickling or smearing over the limits of the substrate or going under the substrate due to lack of glue or due to the adhesive's excessive elasticity.

17. The method of using of the compound for drawing a non-permanent tattoo according to claim 12, comprising the provision of adhesive protective backings which maintain the compound's humidity when applied upon the skin.

18. The method of using of the compound for drawing a non-permanent tattoo according to claim 17, wherein said protective backings act during the method of application to increase the temperature of the region where the tattoo is being applied.

19. The method of using of the compound for drawing a non-permanent tattoo according to claim 18, wherein the increase in temperature increases the reaction capacity of the compound with the skin.

20. The method of using of the compound for drawing a non-permanent tattoo according to claim 17, wherein said backings protects the tattooed skin area avoiding the tattoo displacement and avoiding the fact that the compound contacts other parts of the body.

21. The method of using of the compound for drawing a non-permanent tattoo according to claim 17, wherein said protective backings additionally ensure the uniformity in colour intensity of the tattoo being applied.

22. The method of using of the compound for drawing a non-permanent tattoo according to claim 17, wherein said compound uses the limits of the Genipa americana fruit juice's concentration from 98% down to 10% of coloring strength, for variable coloration intensities.

23. The method of using of the compound for drawing a non-permanent tattoo according to claim 22, wherein when used in a lower concentration the said compound makes it possible to obtain shades of a lighter blue color and when used in a higher concentration makes it possible to obtain shades of black and navy blue colors.

24. The method of using of the compound for drawing a non-permanent tattoo according to claim 17, wherein said compound is in the form of a cream which allows the trace to be done in precisely fine lines.
